(19) United States
(12) Patent Application Publication
(10) Pub. No.: US 2025/0319232 A1
(43) Pub. Date: Oct. 16, 2025

(54) SYSTEM AND METHODS FOR SOFT TISSUE REPAIR STEM GRAFTS

(71) Applicant: TheraMicro, Memphis, TN (US)

(72) Inventors: Alan G. Taylor, Memphis, TN (US); Rebecca Hawkins Wahl, Escondido, CA (US)

(73) Assignee: TheraMicro, Memphis, TN (US)

(21) Appl. No.: 19/080,696

(22) Filed: Mar. 14, 2025

Related U.S. Application Data
(60) Provisional application No. 63/565,951, filed on Mar. 15, 2024.

(51) Int. Cl. A61L 27/36; A61L 27/40

(52) U.S. Cl. CPC A61L 27/3604; A61L 27/40; A61L 2430/34

(57) ABSTRACT

A system and methods are provided for amniotic grafts for encouraging healing of incisions applied to the feet, ankles, and other parts of the body. The amniotic graft comprises a multi-layer matrix and a shape for treating the incisions. The amniotic graft comprises an acellular amniotic membrane or a cellular amniotic membrane. The multi-layer matrix comprises an amnion or chorion layer that enables rapid remodeling of soft-tissue wounds. A spongy layer separates a fibroblast layer and the amnion or chorion layer. The multi-layer matrix includes a compact layer, a basement membrane, and an epithelium. The shape comprises a rectangle having a width and length that are suitable for treating the incision. The amniotic grafts can be sterile packaged with sterile instruments for implanting the grafts to treat soft-tissue wounds. The wound can include surgical incisions, muscle strains, as well as tendon and ligament disruption to various locations of the body.

### PRIORITY

This application claims the benefit of and priority to U.S. Provisional application, entitled “System And Methods For A Soft Tissue Repair Stem Strips,” filed on Mar. 15, 2024, and having application Ser. No. 63/565,951, the entirety of said application being incorporated herein by reference.

### FIELD

Embodiments of the present disclosure generally relate to soft tissue repair. More specifically, embodiments of the disclosure relate to a system and methods for soft tissue grafts for healing of incisions applied to the feet, ankles, and other parts of the body.

### BACKGROUND

Soft tissue injuries can take a long time to heal. Current state of the art are amniotic tissue grafts which are placed on a soft tissue injury to help promote healing. Amniotic grafts contain stem cells which studies have shown to positively impact healing of soft tissue injuries. The grafts are typically supplied in the form of patches having large square shapes. Surgeons are required to cut the patches to the desired shape and carefully place them. The grafts are thin and difficult to handle. What is needed, therefore, is a formulation of preferred sizes and shapes of amniotic grafts and a delivery method to facilitate their placement.

### SUMMARY

A system and methods are provided for amniotic grafts for encouraging healing of incisions applied to the feet, ankles, and other parts of the body. The amniotic graft comprises a multi-layer matrix and a shape for treating the incisions. The amniotic graft comprises an acellular amniotic membrane or a cellular amniotic membrane. The multi-layer matrix comprises an amnion or chorion layer that enables rapid remodeling of soft-tissue wounds. A spongy layer separates a fibroblast layer and the amnion or chorion layer. The multi-layer matrix includes a compact layer, a basement membrane, and an epithelium. The shape comprises a rectangle having a width and length that are suitable for treating the incision. The amniotic grafts can be sterile packaged with sterile instruments for implanting the grafts to treat soft-tissue wounds. The wound can include surgical incisions, muscle strains, as well as tendon and ligament disruption to various locations of the body.

In an exemplary embodiment, an apparatus for encouraging soft-tissue healing comprises: an amniotic graft comprising a multi-layer matrix; and a shape of the amniotic graft for treating a soft-tissue wound.

In another exemplary embodiment, the soft-tissue wound comprises a surgical incision performed on a human foot or an ankle. In another exemplary embodiment, the shape comprises a rectangle having a width and a length. In another exemplary embodiment, the width ranges from about 3 mm to about 10 mm. In another exemplary embodiment, the length ranges from about 6 mm to about 120 mm.

In another exemplary embodiment, the multi-layer matrix comprises an amnion or chorion layer that enables rapid remodeling of the soft-tissue wound. In another exemplary embodiment, the multi-layer matrix includes a spongy layer that separates a fibroblast layer and the amnion or chorion layer. In another exemplary embodiment, the multi-layer matrix comprises a compact layer, a basement membrane, and an epithelium. In another exemplary embodiment, the amniotic graft comprises either an acellular amniotic membrane or a cellular amniotic membrane.

In an exemplary embodiment, a sterile kit for encouraging soft-tissue healing comprises: at least one amniotic graft having a suitable shape for treating a soft-tissue wound; and at least one instrument for implanting the at least one amniotic graft.

In another exemplary embodiment, the at least one instrument comprises tweezers or forceps for clasping the at least one amniotic graft. In another exemplary embodiment, the soft-tissue wound comprises a surgical incision performed on a human foot or an ankle. In another exemplary embodiment, the suitable shape comprises a rectangle having a width and a length. In another exemplary embodiment, the width ranges from about 3 mm to about 10 mm. In another exemplary embodiment, the length ranges from about 6 mm to about 120 mm.

In another exemplary embodiment, the at least one amniotic graft comprises a multi-layer matrix formed from amnion or chorion of an amniotic membrane. In another exemplary embodiment, the multi-layer matrix comprises an amnion or chorion layer that enables rapid remodeling of wounds. In another exemplary embodiment, the multi-layer matrix includes a spongy layer that separates a fibroblast layer and the amnion or chorion layer. In another exemplary embodiment, the multi-layer matrix comprises a compact layer, a basement membrane, and an epithelium. In another exemplary embodiment, the at least one amniotic graft comprises either an acellular amniotic membrane or a cellular amniotic membrane.

These and other features of the concepts provided herein may be better understood with reference to the drawings, description, and appended claims.

### BRIEF DESCRIPTION OF THE DRAWINGS

The drawings refer to embodiments of the present disclosure in which:

FIG. 1 illustrates an exemplary-use environment wherein an exemplary embodiment of an amniotic graft is applied to an incision by use of an instrument, according to the present disclosure;

FIG. 2 illustrates an exemplary embodiment of an amniotic graft being clasped by way of a suitable instrument in accordance with the present disclosure; and

FIG. 3 illustrates an exemplary embodiment of a multi-layer matrix that may be incorporated into amniotic grafts, according to the present disclosure.

While the present disclosure is subject to various modifications and alternative forms, specific embodiments thereof have been shown by way of example in the drawings and will herein be described in detail. The present disclosure should be understood to not be limited to the particular forms disclosed, but on the contrary, the intention is to cover all modifications, equivalents, and alternatives falling within the spirit and scope of the present disclosure.

### DETAILED DESCRIPTION

In the following description, numerous specific details are set forth in order to provide a thorough understanding of the present disclosure. It will be apparent, however, to one of ordinary skill in the art that the amniotic grafts and methods disclosed herein may be practiced without these specific details. In other instances, specific numeric references such as “first instrument,” may be made. However, the specific numeric reference should not be interpreted as a literal sequential order but rather interpreted that the “first instrument” is different than a “second instrument.” Thus, the specific details set forth are merely exemplary. The specific details may be varied from and still be contemplated to be within the spirit and scope of the present disclosure. The term “coupled” is defined as meaning connected either directly to the component or indirectly to the component through another component. Further, as used herein, the terms “about,” “approximately,” or “substantially” for any numerical values or ranges indicate a suitable dimensional tolerance that allows the part or collection of components to function for its intended purpose as described herein.

Soft tissue injuries can take a long time to heal. Current state of the art are amniotic tissue grafts which are placed on a soft tissue injury to help promote healing. Amniotic grafts contain stem cells which studies have shown to positively impact healing of soft tissue injuries. The grafts are typically supplied in the form of patches having large square shapes. Surgeons are required to cut the patches to the desired shape and carefully place them. The grafts are thin and difficult to handle. Embodiments in the present disclosure provide amniotic graft formulated as solid shaped patches that are shaped to be well-adapted to fit in open wounds and incision sites primarily in the foot and ankle.

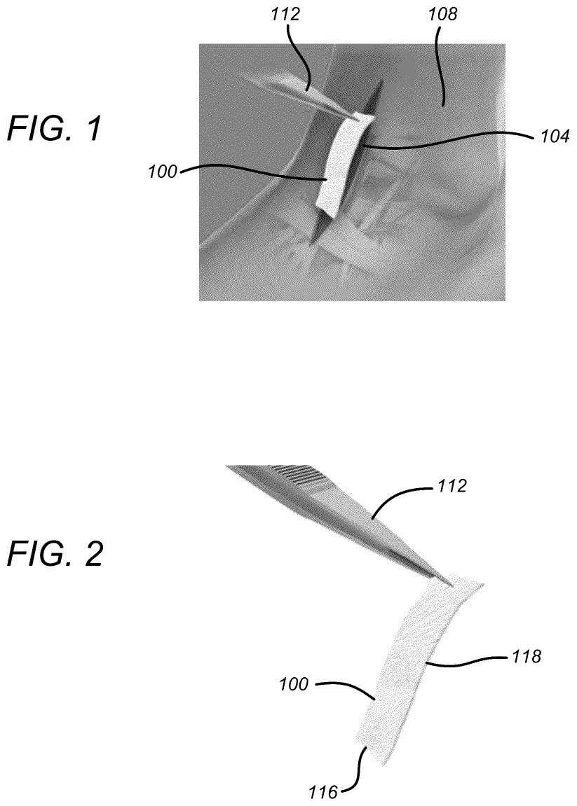

FIG. 1 illustrates an exemplary-use environment wherein an exemplary embodiment of an amniotic graft 100 is being applied to an incision 104 in a foot 108 by use of an instrument 112, according to the present disclosure. In the illustrated embodiment, the amniotic graft 100 comprises a strip shape that is suitable for application of the incision 104. Although the illustrated incision 104 is shown disposed in the foot 108, the incision 104 can, in some embodiments, include any of surgical incisions, wounds, muscle strains, as well as tendon and ligament disruption to various locations of the body.

Moreover, wounds that may be treated by way of the amniotic graft 100 are contemplated to include any one or more of partial or full thickness wounds, pressure sores or ulcers, venous ulcers, diabetic ulcers, chronic vascular ulcers, tunneled or undermined wounds, surgical wounds, trauma wounds (e.g., abrasions, lacerations, partial thickness burns, skin tears), and draining wounds, without limitation.

In general, the shape of the amniotic graft 100 may be specifically configured to facilitate coverage of surgical incisions, such as the incision 104, to promote healing. For example, FIG. 2 illustrates an exemplary embodiment of a strip-shaped amniotic patch 100 being clasped by way of a suitable instrument 112 in accordance with the present disclosure. In some embodiments, the amniotic graft 100 can include rectangular shapes having widths 116 that range from about 3 mm to about 10 mm and have lengths 118 ranging from about 6 mm to about 120 mm, without limitation.

It is contemplated that the amniotic grafts 100 can be supplied in sterile kits that include one or more sterile instruments, such as the instrument 112. The instrument 112 can be any device that is found to facilitate implanting the amniotic graft 100, such as tweezers, forceps, or something similar, without limitation.

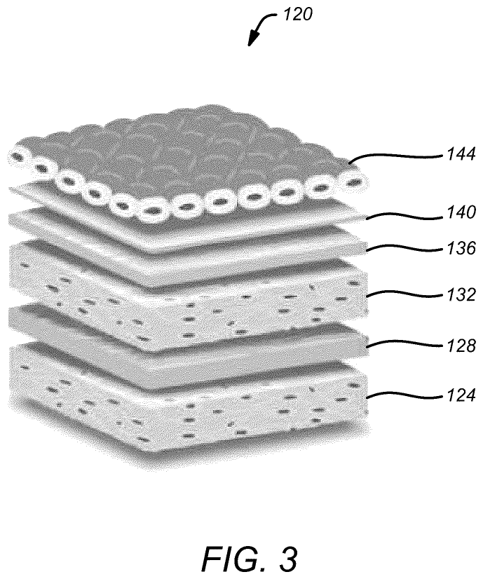

FIG. 3 illustrates an exemplary embodiment of a multi-layer matrix 120 that may be incorporated into an amniotic graft 100, according to the present disclosure. In general, the amniotic graft 100 material can be formed from amnion or chorion of the amniotic membrane. As such, the amniotic graft 100 is a growth factor-rich allograft comprising an amnion or chorion layer 124 that enables rapid remodeling of wounds. As shown in FIG. 3, a spongy layer 128 separates a fibroblast layer 132 and the amnion or chorion layer 124. Further, the multi-layer matrix 120 comprises a compact layer 136, a basement membrane 140 and an epithelium 144. The amniotic graft 100 can be either acellular or cellular amniotic membrane.

While the amniotic grafts and methods have been described in terms of particular variations and illustrative figures, those of ordinary skill in the art will recognize that the amniotic grafts are not limited to the variations or figures described. In addition, where methods and steps described above indicate certain events occurring in certain order, those of ordinary skill in the art will recognize that the ordering of certain steps may be modified and that such modifications are in accordance with the variations of the amniotic grafts. Additionally, certain of the steps may be performed concurrently in a parallel process, when possible, as well as performed sequentially as described above. To the extent there are variations of the amniotic grafts, which are within the spirit of the disclosure or equivalent to the amniotic grafts found in the claims, it is the intent that this patent will cover those variations as well. Therefore, the present disclosure is to be understood as not limited by the specific embodiments described herein, but only by scope of the appended claims.

What is claimed is:

1. An apparatus for encouraging soft-tissue healing, comprising:
an amniotic graft comprising a multi-layer matrix; and
a shape of the amniotic graft for treating a soft-tissue wound.

2. The apparatus of claim 1, wherein the soft-tissue wound comprises a surgical incision performed on a human foot or an ankle.

3. The apparatus of claim 1, wherein the shape comprises a rectangle having a width and a length.

4. The apparatus of claim 3, wherein the width ranges from about 3 mm to about 10 mm.

5. The apparatus of claim 3, wherein the length ranges from about 6 mm to about 120 mm.

6. The apparatus of claim 1, wherein the multi-layer matrix comprises an amnion or chorion layer that enables rapid remodeling of the soft-tissue wound.

7. The apparatus of claim 6, wherein the multi-layer matrix includes a spongy layer that separates a fibroblast layer and the amnion or chorion layer.

8. The apparatus of claim 7, wherein the multi-layer matrix comprises a compact layer, a basement membrane, and an epithelium.

9. The apparatus of claim 8, wherein the amniotic graft comprises either an acellular amniotic membrane or a cellular amniotic membrane.

10. A sterile kit for encouraging soft-tissue healing, comprising:
at least one amniotic graft having a suitable shape for treating a soft-tissue wound; and
at least one instrument for implanting the at least one amniotic graft.

11. The sterile kit of claim 10, wherein the at least one instrument comprises tweezers or forceps for clasping the at least one amniotic graft.

12. The sterile kit of claim 10, wherein the soft-tissue wound comprises a surgical incision performed on a human foot or an ankle.

13. The sterile kit of claim 10, wherein the suitable shape comprises a rectangle having a width and a length.

14. The sterile kit of claim 13, wherein the width ranges from about 3 mm to about 10 mm.

15. The sterile kit of claim 13, wherein the length ranges from about 6 mm to about 120 mm.

16. The sterile kit of claim 10, wherein the at least one amniotic graft comprises a multi-layer matrix formed from amnion or chorion of an amniotic membrane.

17. The sterile kit of claim 16, wherein the multi-layer matrix comprises an amnion or chorion layer that enables rapid remodeling of wounds.

18. The sterile kit of claim 17, wherein the multi-layer matrix includes a spongy layer that separates a fibroblast layer and the amnion or chorion layer.

19. The sterile kit of claim 18, wherein the multi-layer matrix comprises a compact layer, a basement membrane, and an epithelium.

20. The sterile kit of claim 16, wherein the at least one amniotic graft comprises either an acellular amniotic membrane or a cellular amniotic membrane.
